(19) United States
(12) Patent Application Publication
(10) Pub. No.: US 2002/0187210 A1
(43) Pub. Date: Dec. 12, 2002

(54) Method of treating chest pain

(76) Inventor: Carl E. Hanson, Lanark Village, FL (US)

Correspondence Address: Karl G. Hanson, PO Box 33427, St. Paul, MN, 55133-3427, US

(21) Appl. No.: 10/214,253

(22) Filed: Aug. 6, 2002

Related U.S. Application Data
(63) Continuation-in-part of application No. 08/903,677, filed on Jul. 31, 1997, now Pat. No. 6,457,474.

(51) Int. Cl. A61K 35/78

(52) U.S. Cl. 424/736

(57) ABSTRACT

A method of alleviating chest pain that stems from the heart, which method comprises: (a) noticing a pain in the chest; and then shortly thereafter (b) taking a pill that contains lime juice orally to alleviate the chest pain.

This is a continuation-in-part of U.S. patent application Ser. No. 08/903,677 filed Jul. 31, 1997. The disclosure of that application is incorporated into this document by reference in its entirety.

This invention pertains to a method of treating chest pain or angina pectoris.

### BACKGROUND

Angina pectoris is a condition where a person feels paroxysmal pain in the chest. This clinical complex is characterized by various degrees of chest pain that occurs in sudden attacks. The chest pain may be accompanied by other symptoms, notably pain or discomfort of the arms, shoulders, and other sites. The symptoms are most often induced by some physical or emotional stress and often subside promptly with rest or appropriate therapy. McGraw Hill Encyl. Sci. & Tech., vol. 1, p. 539 (6^{th} Ed. 1987).

The chest pain typically is associated with an insufficient supply of blood to a portion of the heart. Common occurrences that precede an angina attack are changes that cause a decrease in blood supply to heart muscle or that create sudden extra demands on the heart so that there is a relative inadequacy of blood. Arteriosclerosis frequently is responsible for the narrowing or partial occlusion of one of the coronary arteries or branches, but other contributing factors such as diabetes mellitus, familial incidence, and emotional stress are examples of other disorders that may set the stage for an anginal attack. This form of coronary heart disease sometimes is a precursor to a heart attack or coronary thrombosis, where a persons artery becomes occluded by a blood clot. There is much overlap between angina pectoris and other forms of coronary heart disease such as arteriosclerotic heart disease and myocardial infarction. McGraw Hill Encyl. Sci. & Tech., vol. 8, p. 346 (6^{th} Ed. 1987).

Doctors regularly prescribe nitroglycerin to patients that suffer from angina pectoris. After oral administration, nitroglycerin is metabolized rapidly in the intestinal wall and liver, so that systemic bioavailability is rather low. Consequently, oral doses are quite high and plasma levels are erratic. Medical authorities do not recommend sustained-release forms of nitroglycerin because of poor oral bioavailability and favorable tolerances. Remington's Pharmaceutical Sciences, Chap. 41, p. 844, (18^{th} Ed. 1990).

Nitroglycerin is commonly administered in the form of very small tablets that are approximately one-eight inch in diameter. These tablets are very difficult for the patient to handle: they can be easily dropped and often more than one tablet comes out of the bottle during dispensing. Size also makes it hard for the patient to return unneeded tablets to the bottle, particularly when moisture is present on the patient's hand.

When taking nitroglycerin, patients put the tablet under their tongue and wait about two-to-four minutes for the tablet to dissolve. It sometimes is not easy for the patient to ascertain if the medicine has been properly ingested. The patient then waits approximately five minutes for the angina attack to go away.

### SUMMARY OF THE INVENTION

This invention provides an alternative to taking nitroglycerin to combat chest pain such as angina pectoris. In accordance with this invention, a person takes a pill that contains lime juice after noticing the onset of the chest pain. As the term is used in this document, “lime juice” means lime juice or limeade or any combination that includes the juice from a lime in concentrated or diluted form as a solid or liquid, or that includes the active ingredients in the lime that counter the chest pain or anginal attack. The term “pill” means a tablet, pellet, or capsule sized to be manually handled for oral intake by a person.

The inventor surprisingly discovered that, by taking a pill that contains lime juice shortly after noticing that he was experiencing a chest pain, the pain immediately subsided. The pill can be easily administered by, for example, placing it in the mouth, letting it dissolve or chewing it, and then swallowing. The pill also could be swallowed whole if desired. The inventor believes that by consuming such pills regularly, for example, that the recurrence of chest pain can be substantially reduced.

The present invention is advantageous in that a patient can easily determine if the medicine is properly ingested. Limes have a very noticeable taste that disappears after it leaves the mouth. Since limes are regularly available and are naturally occurring, pills of the invention may be fabricated without involving relatively complex synthetic production techniques. Additionally, patients should experience no side effects unless they happen to be allergic to limes.

These and other advantages of the invention are more fully described below in the detailed description of this invention.

### DETAILED DESCRIPTION OF PREFERRED EMBODIMENTS

In practicing the present invention, a person who experiences or has experienced chest pain associated with a heart condition takes a pill that contains lime juice to alleviate the pain. The inventor discovered that after ingesting at least one pill, that his chest pain subsided remarkably. The inventor found it much less troublesome to ingest the lime juice pills as opposed to taking the very small nitroglycerin tablets because the inventive pills are easy to handle. After learning of the benefits of lime juice, the inventor used the pills solely without taking nitroglycerin and without fear that the chest pains would reoccur.

Each pill generally contains at least about 0.1 teaspoon of unconcentrated, nondiluted lime juice. The lime juice is typically in liquid form during the manufacturing process but may be in dried or solid form when present in the pills. The lime juice may be added in amounts as high as 10 teaspoons of liquid per pill or higher during processing. The volume, of course, decreases upon drying. Typically, about 0.25 to 5 teaspoons of unconcentrated, nondiluted liquid lime juice are employed in the mixture that forms the pills. More typically, at least about 0.5 to 3 teaspoons are used per pill when preparing the mixture that is subsequently dried to create the pills. The amounts of lime juice, which is described in this paragraph, refer to “pure lime juice”, which is the liquid product extracted from the lime core. The limes that may be used in this invention may be obtained from, for example, the Caribbean, Mexico, and other locations. Limes from Haiti have been found to give good results. In forming the pills, the lime juice is preferably employed with inert ingredients that allow a solid pill that has sufficient structural integrity to be produced from the mixture. As the term is used in this application, “inert ingredients” are those that do not significantly effect the lime juice's ability to alleviate chest pain. Preferred inert ingredients also do not cause serious side effects amongst a significant percentage of persons. The lime juice can, for example, be mixed with corn starch and water, placed on a substrate such as a sheet, tray, or mold, and then be allowed to dry. The pills, if desired, may have a covering that prevents them from sticking to the fingers and that helps maintain the pills' structural integrity. Sweeteners, such as honey, could also be added to the pills in conjunction with the lime juice. Capsules also could be used, which contain the lime juice in solid and/or liquid form, by itself or in conjunction with optional inert ingredients.

The pill may be taken in amounts suitable to each individual who experiences cardiac chest pain. Generally, at least one pill should be taken for each occurrence of chest pain. More pills, however, may be taken to ensure that the pain does not recur. To this end, at least one pill may be taken daily. If regular, more pills, for example, 2 to 10 daily, may be taken by the person experiencing the chest pain.

The following Examples have been selected to further illustrate features, advantages, and other details of the invention. It is to be expressly understood, however, that while the Example serve this purpose, the particular ingredients and amounts used as well as other conditions and details are not to be construed in a manner that would unduly limit the scope of this invention.

### EXAMPLE

### Example 1

I purchased a half dozen conventional whole limes from the IGA grocery store in Carabelle, Fla. The limes were cut in half-inch pieces, and then the pieces were put in a 300 watt Tyron™ electric juice extractor. The juice extractor separates the rind and some pulp from the juice. Cornstarch (1 tablespoon) was added to one half cup of water. The cornstarch and water was slightly heated and mixed to get a smooth, thick consistency. Then a quarter cup of lime juice was added to the warm thickened mixture as it cooled. Half-teaspoon samples of the mixture were placed on plastic wrap and were allowed to cool to form about a dozen pills about a half-inch in diameter.

Upon noticing the onset of chest pain, I took one of the pills. I noticed a response but not a complete response. Approximately two minutes later, I took two more of the pills and then the chest pain disappeared.

### Example 2

The steps of Example 1 were repeated, except the amounts of lime juice and other ingredients were approximately doubled. A teaspoon of green food coloring was also added so that the resulting pills would display a green color. Tablespoon size samples of the mixture were placed in paper cupcake forms. The samples were allowed to cool to create approximately a half dozen one-inch diameter tablets or pills.

Upon noticing the onset of chest pain, I took one of the pills and the pain disappeared in approximately two to three minutes. The pain subsided for at least one day.

What is claimed is:

1. A method of preventing the reoccurrence of chest pain associated with the heart, which method comprises:
(a) noticing a pain in the chest; and then shortly thereafter
(b) orally administering a pill that contains lime juice to alleviate the chest pain.

2. The method of claim 1, wherein the chest pain is angina pectoris.

3. The method of claim 2, wherein the pill contains at least about 0.1 teaspoon of unconcentrated, nondiluted lime juice per pill.

4. The method of claim 2, wherein the pill contains approximately 0.25 to 5 teaspoons of unconcentrated, nondiluted lime juice per pill.

5. The method of claim 2, wherein the pill contains approximately 0.5 to 3 teaspoons of lime juice per pill.

6. The method of claim 1, further comprising:
preventing the reoccurrence of chest pain by orally taking the pills daily.

7. The method of claim 6, wherein at least one pill is consumed orally daily.

8. The method of claim 7, wherein 1 to 10 pills are consumed daily.

9. A method of treating angina pectoris, which method comprises:
(a) noticing the onset of an angina attack; and then shortly thereafter
(b) orally administering a pill that contains lime juice.

10. The method of claim 1, wherein the pills comprise lime juice and inert ingredients.

11. The method of claim 10, wherein the pills also contain honey.

12. A method of alleviating chest pain that stems from the heart, treating angina pectoris which method comprises:
orally administering a pill that comprises lime juice in dried form and inert ingredients to a person shortly after experiencing chest pain.

13. The method of claim 12, wherein the pill was formed from a mixture that comprises 0.1 to 10 teaspoons of pure lime juice per and inert ingredients.

14. The method of claim 12, wherein the pill displays a green color.

15. A pill that is for alleviating cardiac chest pain, the pill comprising lime juice as an active ingredient for alleviating the pain.

16. The pill of claim 15, further comprising inert ingredients.

17. The pill of claim 16 being in the form of a tablet.

18. The pill of claim 15 being in the form of a capsule.

19. The pill of claim 15, comprising 0.25 to 10 teaspoons of lime juice per pill.

20. The pill of claim 15, comprising 0.5 to 2 teaspoons of lime juice per pill.
